(19) United States
(12) Patent Application Publication
(10) Pub. No.: US 2011/0311521 A1
(43) Pub. Date: Dec. 22, 2011

(54) Novel therapy for anxiety

(76) Inventors: Pico Caroni, Muttenz (CH); Nadine Gogolla, Cambridge, MA (US); Cyril Herry, Cabanac (FR); Andreas Luethi, Basel (CH)

(21) Appl. No.: 13/254,639

(22) PCT Filed: Mar. 5, 2010
(86) PCT No.: PCT/EP10/52793

§ 371 Date: Sep. 2, 2011

(30) Foreign Application Priority Data
Mar. 6, 2009 (EP) 09154488.2

(51) Int. Cl. A61K 39/395; A61K 31/7088; A61K 38/43; C12N 9/24; C07K 16/18; A61K 38/51; A61P 25/22; A61K 38/02; C07H 21/02

(52) U.S. Cl. 424/130.1; 514/17.5; 514/44 A; 424/94.1; 536/24.5; 530/389.1; 424/94.5; 435/200

(57) ABSTRACT

The present invention relates to a method of modulating, treating or effecting prophylaxis of a subject having anxiety or at risk of developing symptoms of anxiety, said method being characterized in that a therapeutically effective amount of a modulator of chondroitin sulphate proteoglycans is administered to said subject.

### FIELD OF THE INVENTION

The present invention relates to a method of modulating, treating or effecting prophylaxis of a subject having anxiety or at risk of developing symptoms of anxiety.

### BACKGROUND OF THE INVENTION

Anxiety and depression are major psychiatric disorders of significant clinical and socioeconomic significance. Clinical Depression often presents alongside Anxiety Disorders, and vice-versa. The co-morbidity of Anxiety Disorders and Depression is a frequent therapeutical challenge. In the general population, these disorders affect daily performance and correlate with impulse control, financial behaviors, substance abuse and organization. Anxiety is an unpleasant state that involves a complex combination of emotions that include fear, apprehension, and worry. It is often accompanied by physical sensations such as heart palpitations, nausea, chest pain, shortness of breath, or tension headache. Anxiety disorder is a blanket term covering several different forms of abnormal, pathological anxiety, fears, phobias and nervous conditions that may come on suddenly (acute anxiety) and/or gradually over a period of several years (chronic), and may impair or prevent the pursuing of normal daily routines. Anxiety disorders are often debilitating chronic conditions, which can be present from an early age or begin suddenly after a triggering event. They are prone to flare up at times of high stress.

Anxiety is often described as having cognitive, somatic, emotional, and behavioral components (Seligman, Walker & Rosenhan, 2001). The cognitive component entails expectation of a diffuse and uncertain danger. Somatically the body prepares the organism to deal with threat (known as an emergency reaction): blood pressure and heart rate are increased, sweating is increased, bloodflow to the major muscle groups is increased, and immune and digestive system functions are inhibited. Externally, somatic signs of anxiety may include pale skin, sweating, trembling, and pupillary dilation. Emotionally, anxiety causes a sense of dread or panic and physically causes nausea, and chills. Behaviorally, both voluntary and involuntary behaviors may arise directed at escaping or avoiding the source of anxiety. These behaviors are frequent and often maladaptive, being most extreme in anxiety disorders. However, anxiety is not always pathological or maladaptive: it is a common emotion along with fear, anger, sadness, and happiness, and it has a very important function in relation to survival. Conventional treatments for anxiety include behavioral therapy, lifestyle changes and/or pharmaceutical therapy (medications). Most drugs used to treat these disorders are known to have negative side effects that may limit their use, or cause habituation and dependence.

There is converging evidence that anxiety disorders and depression share common neurobiological substrates involving dysfunction of the amygdala and its interactions with the prefrontal cortex and the hippocampus (Phillips et al., 2003; Drevets et al., 2008). Classical fear conditioning is a powerful translational animal model widely used to study the neuronal substrates underlying anxiety disorders in humans and animals (Phelps and LeDoux, 2005).

In classical fear conditioning, pairing an initially neutral stimulus (conditioned stimulus; CS), with an aversive stimulus (unconditioned stimulus; US) leads to the formation of a robust and long-lasting fear memory (1). In rats, such memories can last for the entire lifetime (2). Inhibition of conditioned fear responses can be achieved by repeated exposure to the CS in the absence of the US, a process called extinction (3). In contrast to fear conditioning, fear extinction is neither robust nor permanent. After extinction training, conditioned fear responses can recover spontaneously, following re-exposure to the US (reinstatement), or in response to a context-shift (renewal) (3-5). This strongly indicates that fear extinction does not erase previously acquired fear memories, but involves new learning eventually inhibiting conditioned fear behavior.

Extinction of conditioned fear depends on a distributed and highly interconnected neuronal network comprising the amygdala, the medial prefrontal cortex and the hippocampus (3-5). During development, these connections form relatively late. In rats, amygdala-mPFC connectivity is not fully established until several weeks after birth (6, 7). Since young postnatal rats can acquire and extinguish conditioned fear responses well before (8, 9), the present inventors concluded that the neuronal mechanisms underlying fear extinction during early development must fundamentally differ from those operating in adults. Indeed, 24 hours after extinction training, rats younger than three weeks do not exhibit reinstatement or context-dependent renewal of conditioned fear responses (9, 10), yet extinction still depends on the amygdala (11). During early development, extinction thus appears to be permanent, and has been suggested to reflect an unlearning process leading to the erasure of previously conditioned fear memories (11). The neuronal mechanisms underlying the developmental regulation of fear extinction are, however, not known.

Developmental regulation of brain plasticity is much better understood in sensory systems, such as the visual cortex. During the first few weeks of postnatal development, the so-called critical period, monocular sensory deprivation leads to long-lasting functional and structural changes (12). The low and diffuse expression of chondroitin sulphate proteoglycans (CSPGs), an important component of the brain extracellular matrix (13), is considered to be a key permissive factor allowing for the induction of ocular dominance plasticity during the critical period. Expression of CSPGs reaches adult levels by the time of critical period closure (14). In addition, the degradation of CSPGs in adults re-enables the induction of ocular dominance plasticity (14).

### SUMMARY OF THE INVENTION

The present inventors have now surprisingly found that whereas CSPGs are not required for the acquisition, retrieval and expression of conditioned fear memory, extinction training triggers in the absence of CSPGs a rapid process that results in the acute and permanent loss of conditioned fear behavior. In other words, the present inventors have now surprisingly found that degradation of CSPGs in adult mice phenocopies the behavioral consequences of fear extinction in young postnatal animals, and showed that CSPGs prevent unlearning or erasure of fear memories in adults

The present invention therefore encompasses a method of modulating, treating or effecting prophylaxis of a subject having anxiety or at risk of developing symptoms of anxiety, said method being characterized in that a therapeutically effective amount of a modulator of chondroitin sulphate proteoglycans is administered to said subject. In some embodiments of the method of the invention, the modulator degrades the chondroitin sulphate proteoglycans of the extracellular matrix. In some other embodiments of the methods of the invention, the modulator modulates the expression, translation and/or secretion of the chondroitin sulphate proteoglycans to the extracellular matrix. In yet other embodiments of the method of the invention, the modulator decreases or increases the interaction of the chondroitin sulphate proteoglycans of the extracellular matrix with their counterparts, and/or modifies the molecular organization of the chondroitin sulphate proteoglycans of the extracellular matrix.

In some embodiments of methods of the invention, the modulator of is a small molecule. In other embodiments of the methods of the invention, the modulator is an antibody. In yet other embodiments of the methods of the invention the modulator is a peptide, for example a peptide mimetic of the chondroitin sulphate proteoglycans, and/or Low Molecular Weight Chondroitin Sulfate, that would disturb the formation of the extracellular matrix.

In some embodiments of the methods of the invention, the modulator decreases or silences the expression of chondroitin sulphate proteoglycans or of a negative regulator thereof. For example the modulator is a siRNA, for instance directed against a key enzyme for the biosynthesis of the chondroitin sulphate proteoglycans, for example a glycosyl transferase.

In some embodiments of the invention, the modulator is an enzyme, for instance a chondroitinase.

In some embodiments of the methods of the invention, the subject is a mammal, for instance a human.

In some embodiments of the methods of the invention, the modulator is delivered or applied to the amygdala of the subject, for instance by a pump delivering the modulator though a duct to a pre-defined region, e.g. the amygdala of the subject.

The present invention also encompasses a siRNA modulating the expression, translation and/or secretion of the chondroitin sulphate proteoglycans to the extracellular matrix, an antibody specifically binding to chondroitin sulphate proteoglycans, or an enzyme degrading chondroitin sulphate proteoglycans, for instance a chondroitinase, for use as a medicament to treat of prevent anxiety.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1. Coincident developmental switch in CSPG expression and in the susceptibility of fear memories to erasure.

(A) Experimental protocol. Fear Cond.: fear conditioning; Cxt: context. (B) One week after extinction training, comparison of freezing levels in mice conditioned at postnatal day (P) 16 (n=5) or P23 (n=4) revealed significant spontaneous recovery and context-dependent renewal in the P23, but not in the P16 group (percent of time spent freezing, recall: P16: 14.6±4.9, P23: 45.8±3.2, P<0.01; renewal: P16: 29.6±9.4, P23: 84.8±8.1 P<0.01, two-tailed unpaired t-test). In P23 mice, freezing levels during renewal were significantly greater as compared to extinction recall (P<0.05, two-tailed paired t-test). (C) In the absence of extinction training, P16 animals (n=7) showed stable fear memory 10 days after conditioning (percent of time spent freezing, recall: P16 No Ext: 48.7±7.9, P16 Ext: 14.6±4.9, P<0.05, two-tailed unpaired t-test). (D) WFA staining in the BLA of P16, P21, P23 and P28 mice. Scale bars: 200 μm (top panels), 30 μm (bottom panels). (E) The number of WFA-positive PNNs increased between P16 and P23 (Number of PNNs: P16 (n=7): 3.3±0.4, P23 (n=5): 14.6±0.3, P<0.001, two-tailed unpaired t-test). *P<0.05, **P<0.01, ***P<0.001.

FIG. 2. Degradation of CSPGs in adult mice abolishes spontaneous recovery and context-dependent renewal of conditioned fear.

(A) Experimental protocol. (B) One week after extinction training, vehicle-injected mice (n=10), but not chABC-injected mice (n=11) exhibited spontaneous recovery (measured in the extinction context) and renewal (measured in the fear conditioning context) of conditioned fear responses (percent of time spent freezing, recall: vehicle: 63.5±8.3, chABC: 24.3±6.3, P<0.01; renewal: vehicle: 85.1±6.3, chABC: 45.7±5.9 P<0.01, two-tailed unpaired t-test). (C) ChABC-injected mice (n=13), but not vehicle-injected mice (n=13), exhibited a rapid reduction in freezing levels already during the first day of extinction training (Day 3) (Two way ANOVA, (group X time), group: F_{(1,24)}=34.3, P<0.001, time: F_{(1,5)}=12.5, P<0.001, interaction between group and time: F_{(5,120)}=6.9, P<0.001). Freezing levels between ChABC and vehicle-injected mice were significantly different on the second block of extinction (percent of time spent freezing, second block of extinction: vehicle: 75.1±4.7, chABC: 44.5±6.9, P<0.001; two-tailed paired t-test). At the end of the second day of extinction (Day 3), both groups reached similar levels of extinction (percent of time spent freezing, last block of extinction: vehicle: 28.8±4.1, chABC: 24.1±5.3, P=0.51; two-tailed paired t-test). **P<0.01.

FIG. 3. Degradation of CSPGs does not interfere with fear memory consolidation.

(A) Experimental protocol for consolidation experiments. (B) Degradation of CSPGs did not affect fear memory consolidation as measured 7 days after fear conditioning (percent of time spent freezing, first block: vehicle: 72.8±7.1, n=5, chABC: 62.5±3.8, n=5, P=0.78; two-tailed unpaired t-test), yet rapid extinction was induced in chABC-injected mice exposed to repetitive non-reinforced CSs (One way ANOVA with repeated measures: vehicle: F_{(4,3)}=0.72, P=0.56, chABC: F_{(4,3)}=18.5, P<0.001).

FIG. 4. Degradation of CSPGs does not affect previously acquired fear memories or memory reconsolidation.

(A) Experimental protocol. (B) Rapid extinction only occurred for conditioned fear responses that had been acquired after chABC injection (Fear Cond. (CS2), n=6), but not for those acquired prior to chABC injection (Fear Cond. (CS1), n=9, two way ANOVA, (group X time), group: F_{(1,13)}=9.15, P<0.01, time: F_{(1,3)}=16.87, P<0.001, interaction between group and time: F_{(3,39)}=5.94, P<0.05). (C) Degradation of CSPGs did not affect fear memory reconsolidation as measured by two consecutive tests separated by 24 hrs.

FIG. 5. (A) Coronal sections through the rostro-caudal extent of the amygdala showing the location of bilateral injection sites for vehicle-injected (red circles) and chABC-injected (blue circles) mice shown in FIG. 2. Numbers indicate the anteroposterior coordinates caudal to bregma (1). (B) Example 2b6 staining after chABC injection into the BLA illustrating location of CSPG degradation.

FIG. 6. Twenty four hours after fear conditioning ChABC- and vehicle-injected mice exhibit discriminative fear responses when exposed to the paired CS(CS⁺) and to an explicitly unpaired CS(CS⁻) (percent time spent freezing, vehicle: CS⁻: 25.8±3.6, CS⁺: 71.7±4.7, P<0.001; chABC: CS⁻: 17.9±3, CS⁺: 66.9±4.9 P<0.001, two-tailed paired t-test).

FIG. 7. One week after extinction a subset of vehicle-injected mice (n=3) with low spontaneous recovery of fear exhibited significant fear renewal when exposed to the conditioned CS in the original conditioning context (percent time spent freezing, recall: CS: 31.8±9.4; renewal: 87.6±1.4, P<0.05, two-tailed paired t-test). *P<0.05.

FIG. 8. Coronal sections through the rostro-caudal extent of the amygdala showing the location of bilateral injection sites for vehicle-injected (red circles) and chABC-injected (blue circles) mice shown in FIG. 3. Anteroposterior coordinates caudal to bregma (K. J. B. Franklin, G. Paxinos, The Mouse Brain in Stereotaxic Coordinates. New York, N.Y.: Academic Press, 1997).

FIG. 9. Coronal sections through the rostro-caudal extent of the amygdala showing the location of bilateral injection sites for chABC-injected mice shown in FIG. 4. Numbers indicate the anteroposterior coordinates caudal to bregma. Anteroposterior coordinates caudal to bregma (K. J. B. Franklin, G. Paxinos, The Mouse Brain in Stereotaxic Coordinates. New York, N.Y.: Academic Press, 1997).

FIG. 10. Normal extinction, spontaneous recovery and renewal in mice injected with chABC after the acquisition of conditioned fear.

(A) Experimental protocol. (B) Mice were injected with chABC immediately after conditioning and submitted to fear extinction. At the end of extinction (Day 3), fear levels were significantly reduced (percent of time spent freezing, Post-FC: 62.5±6.3, Extinction: 9.2±0.3, P<0.05; two-tailed paired t-test). One week later, chABC-injected mice (n=3) exhibited spontaneous recovery and renewal of conditioned fear responses when tested in the extinction and fear conditioning context, respectively (percent of time spent freezing, recall: 36±3.9; renewal: 58±6.4, P<0.05, two-tailed paired t-test). *P<0.05.

### DETAILED DESCRIPTION OF THE INVENTION

The present inventors have now surprisingly found that whereas CSPGs are not required for the acquisition, retrieval and expression of conditioned fear memory, extinction training triggers in the absence of CSPGs a rapid process that results in the acute and permanent loss of conditioned fear behavior. In other words, the present inventors have now surprisingly found that degradation of CSPGs in adult mice phenocopies the behavioral consequences of fear extinction in young postnatal animals, and showed that CSPGs prevent unlearning or erasure of fear memories in adults

The present invention therefore encompasses a method of modulating, treating or effecting prophylaxis of a subject having anxiety or at risk of developing symptoms of anxiety, said method being characterized in that a therapeutically effective amount of a modulator of chondroitin sulphate proteoglycans is administered to said subject. In some embodiments of the method of the invention, the modulator degrades the chondroitin sulphate proteoglycans of the extracellular matrix. In some other embodiments of the methods of the invention, the modulator modulates the expression, translation and/or secretion of the chondroitin sulphate proteoglycans to the extracellular matrix. In yet other embodiments of the method of the invention, the modulator decreases or increases the interaction of the chondroitin sulphate proteoglycans of the extracellular matrix with their counterparts, and/or modifies the molecular organization of the chondroitin sulphate proteoglycans of the extracellular matrix.

In some embodiments of methods of the invention, the modulator of is a small molecule. In other embodiments of the methods of the invention, the modulator is an antibody. In yet other embodiments of the methods of the invention the modulator is a peptide, for example a peptide mimetic of the chondroitin sulphate proteoglycans, and/or Low Molecular Weight Chondroitin Sulfate, that would disturb the formation of the extracellular matrix.

In some embodiments of the methods of the invention, the modulator decreases or silences the expression of chondroitin sulphate proteoglycans or of a negative regulator thereof. For example the modulator is a siRNA, for instance directed against a key enzyme for the biosynthesis of the chondroitin sulphate proteoglycans, for example a glycosyl transferase.

In some embodiments of the invention, the modulator is an enzyme, for instance a chondroitinase.

In some embodiments of the methods of the invention, the subject is a mammal, for instance a human.

In some embodiments of the methods of the invention, the modulator is delivered or applied to the amygdala of the subject, for instance by a pump delivering the modulator though a duct to a pre-defined region, e.g. the amygdala of the subject.

The present invention also encompasses a siRNA modulating the expression, translation and/or secretion of the chondroitin sulphate proteoglycans to the extracellular matrix, an antibody specifically binding to chondroitin sulphate proteoglycans, or an enzyme degrading chondroitin sulphate proteoglycans, for instance a chondroitinase, for use as a medicament to treat of prevent anxiety.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment”, “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, derivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W.H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty-−1, Joining Penalty-−30, Randomization Group Length=0, Cutoff Score=1, Gap Penalty-−5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty-−1, Joining Penalty=20, Randomization Group Length=0, Cutoff Score=1, Window Size=sequence length, Gap Penalty-−5, Gap Size Penalty-−0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

In one embodiment where one is assaying for the ability to bind or compete with CSGPs for binding to anti-CSGPs antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The term “epitopes,” as used herein, refers to portions of a polypeptide having antigenic or immunogenic activity in an animal, in some embodiments, a mammal, for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno-specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In addition, in the context of the present invention, the term “antibody” shall also encompass alternative molecules having the same function, e.g. aptamers and/or CDRs grafted onto alternative peptidic or non-peptidic frames.

In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues.

Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a CSPG of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity.

Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide).

Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat or prevent anxiety. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions, e.g. lyophilized as known in the art or as described herein.

The invention also provides methods for treating or preventing anxiety in a subject by inhibiting or degrading CSGPs by administration to the subject of an effective amount of an modulatory compound or pharmaceutical composition comprising such modulatory compound. In some embodiments, said modulatory compound is an antibody or an siRNA. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-13 8 (1984)).

Other controlled release systems are discussed in the review by Langer (Science 249:1527-1533 (1990)).

The present invention also provides pharmaceutical compositions for use in the treatment and/or prevention of anxiety by modulating extracellular CSPGs. Such compositions comprise a therapeutically effective amount of a modulatory compound/agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration, or intra-cerebral administration, to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems.

For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for example 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustaIX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid.

The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustaIX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained users knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYL1P.

The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the modulator is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among:
a) a 3′ cap;
b) a 5′ cap,
c) a modified internucleoside linkage; or
d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference.

The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi.

It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesize the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like).

Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product).

Modulators according to any embodiment of the present invention may be used in a monotherapy (e.g. use of siRNAs alone). However it will be appreciated that the modulators may be used as an adjunct, or in combination with other therapies.

The modulators of CSGPs may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the modulator to the target site.

The modulators of CSPGs may be used in a number of ways.

In some embodiments, the modulator of CSPGs is given to the subject before said subject could encounter a traumatic event which could lead to anxiety and which is foreseeable, for instance to a soldier.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection—when used in the brain). The modulators may also be administered by inhalation (e.g. intranasally) or even orally (if appropriate).

The modulators of the invention may also be incorporated within a slow or delayed release device. Such devices may, for example, be inserted near the CNS, and the molecule may be released over weeks or months. Such devices may be particularly advantageous when long term treatment with a modulator of CSPGs is required and which would normally require frequent administration (e.g. at least daily injection).

It will be appreciated that the amount of modulator that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the modulator within the subject being treated.

Optimal dosages to be administered may be determined by those skilled in the art, and will vary with the particular modulator in use, the strength of the preparation, and the mode of administration.

Additional factors depending on the particular subject being treated will result in a need to adjust dosages, including subject age, weight, gender, diet, and time of administration.

When the modulator is a nucleic acid conventional molecular biology techniques (vector transfer, liposome transfer, ballistic bombardment etc) may be used to deliver the modulator to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an modulator of CSPGs may be used for the treatment or prevention of anxiety in the subject, depending upon which specific modulator is used. When the modulator is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the modulator (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

### Anxiety

Unless otherwise apparent from the context, reference to “anxiety” includes any of the forms of anxiety defined in the Diagnostic and Statistical Manual of Mental Disorders, 4th edition (DSM-IV-TR) and/or below. Many of the subtypes of anxiety are characterized by acute episodes (i.e., relatively short periods of relatively numerous and/or intense symptoms and signs of disease separated by relatively long intervals of fewer or less intense symptoms and signs, if any). Often acute episodes are often triggered by a specific event that has occurred or is expected to occur imminently. Anxiety and its subtypes are usually diagnosed by applying a questionaire to determine whether patients meet DSM-IV-TR criteria.

### Generalized Anxiety Disorder

Generalized anxiety disorder is a common chronic disorder that affects twice as many women as men and can lead to considerable impairment (Brawman-Mintzer & Lydiard, J. Clin. Psychiatry, 57 (Suppl. 7):3-8, 1996; Bull. Menninger Clin., 61(2 suppl. A):A66-A94, 1997; J. Clin. Psychiatry, 58(suppl. 3): 16-25, 1997). As the name implies, generalized anxiety disorder is characterized by long-lasting anxiety that is not focused on any particular object or situation. In other words it is unspecific or free-floating. People with this disorder feel afraid of something but are unable to articulate the specific fear. They fret constantly and have a hard time controlling their worries. Because of persistent muscle tension and autonomic fear reactions, they may develop headaches, heart palpitations, dizziness, and insomnia. These physical complaints, combined with the intense, long-term anxiety, make it difficult to cope with normal daily activities.

### Panic Disorder

In panic disorder, a person suffers brief attacks of intense terror and apprehension that cause trembling and shaking, confusion, dizziness, nausea, difficulty breathing, and feelings of impending doom or a situation that would be embarrassing. The American Psychiatric Association (2000) defines a panic attack as fear or discomfort that arises abruptly and peaks in 10 minutes or less, and can occasionally last hours.

Although panic attacks sometimes seem to occur out of nowhere, they generally happen after frightening experiences, prolonged stress, or even exercise. Many people who have panic attacks (especially their first one) think they are having a heart attack and often end up at the doctor or emergency room. Even if the tests all come back normal the person will still worry, with the physical manifestations of anxiety only reinforcing their fear that something is wrong with their body. Heightened awareness (hypervigilance) of any change in the normal function of the human body, will be noticed and interpreted as a possible life threatening illness by an individual suffering from panic attacks.

Normal changes in heartbeat, such as when climbing a flight of stairs will be noticed by a panic sufferer and lead them to think something is wrong with their heart or they are about to have another panic attack. Some begin to worry excessively and even quit jobs or refuse to leave home to avoid future attacks. Panic disorder can be diagnosed when several apparently spontaneous attacks lead to a persistent concern about future attacks.

### Agoraphobia

A common complication of panic disorder is agoraphobia—anxiety about being in a place or situation where escape is difficult or embarrassing (Craske, 2000; Gorman, 2000). The definition of the word has expanded to refer to avoidance behaviors that sufferers often develop. If a sufferer of panic attacks seems to have them while driving, for example, then he or she may avoid driving, which relieves the anxiety, and subsequently makes future driving more difficult, as a result of behavioral reinforcement.

### Phobias

This category involves a strong, irrational fear and avoidance of an object or situation. The person knows the fear is irrational, yet the anxiety remains. Phobic disorders differ from generalized anxiety disorders and panic disorders because there is a specific stimulus or situation that elicits a strong fear response. A person suffering from a phobia of spiders might feel so frightened by a spider that he or she would try to jump out of a speeding car to get away from one.

People with phobias have especially powerful imaginations, so they vividly anticipate terrifying consequences from encountering such feared objects as knives, bridges, blood, enclosed places, certain animals or situations. These individuals generally recognize that their fears are excessive and unreasonable but are generally unable to control their anxiety.

### Social Anxiety Disorder

Social anxiety disorder is also known as social phobia. Individuals with this disorder experience intense fear of being negatively evaluated by others or of being publicly embarrassed because of impulsive acts. Almost everyone experiences “stage fright” when speaking or performing in front of a group. Since occasionally there are artists or performers with social anxiety disorder who are able to perform publicly without significant anxiety, their love of performing and practicing their art may be diminishing their anxiety. But people with social phobias often become so anxious that performance, if they are not natural performers, such as children playing musical instruments from a young age, is out of the question. In fact, their fear of public scrutiny and potential humiliation becomes so pervasive that normal life can become impossible (den Boer 2000; Margolis & Swartz, 2001). Another social phobia is love-shyness, which most adversely affects certain men. Those afflicted find themselves unable to initiate intimate adult relationships (Gilmartin 1987).

### Obsessive-Compulsive Disorder

Obsessive compulsive disorder is a type of anxiety disorder primarily characterized by obsessions and/or compulsions. Obsessions are distressing, repetitive, intrusive thoughts or images that the individual often realizes are senseless. Compulsions are repetitive behaviors that the person feels forced or compelled into doing, in order to relieve anxiety. The OCD thought pattern may be likened to superstitions: if X is done, Y won't happen—in spite of how unlikely it may be that doing X will actually prevent Y, if Y is even a real threat to begin with. A common example of this behavior would be obsessing that one's door is unlocked, which may lead to compulsive constant checking and rechecking of doors. Often the process seems much less logical. For example, the compulsion of walking in a certain pattern may be employed to alleviate the obsession that something bad is about to happen. Lights and other househould items are also common objects of obsession.

### Post-Traumatic Stress Disorder

Post-traumatic stress disorder is an anxiety disorder which results from a traumatic experience, such as being involved in battle, rape, being taken hostage, or being involved in a serious accident. The sufferer may experience flashbacks, avoidant behavior, and other symptoms. Post-traumatic stress disorder (PTSD) is a term for certain severe psychological consequences of exposure to, or confrontation with, stressful events that the person experiences as highly traumatic. Clinically, such events involve actual or threatened death, serious physical injury, or a threat to physical and/or psychological integrity, to a degree that usual psychological defenses are incapable of coping with the impact. It is occasionally called post-traumatic stress reaction to emphasize that it is a result of traumatic experience rather than a manifestation of a pre-existing psychological condition. The presence of a PTSD response is influenced by the intensity of the experience, its duration, and the individual person involved.

PTSD may be triggered by an external factor or factors. Its symptoms can include the following: nightmares, flashbacks, emotional detachment or numbing of feelings (emotional self-mortification or dissociation), insomnia, avoidance of reminders and extreme distress when exposed to the reminders (“triggers”), loss of appetite, irritability, hypervigilance, memory loss (may appear as difficulty paying attention), excessive startle response, clinical depression, and anxiety. It is also possible for a person suffering from PTSD to exhibit one or more other comorbid psychiatric disorders; these disorders often include clinical depression (or bipolar disorder), general anxiety disorder, and a variety of addictions.

Symptoms that appear within the first month of the trauma are called Acute stress disorder, not PTSD according to DSM-IV. If there is no improvement of symptoms after this period of time, PTSD is diagnosed. PTSD has three subforms: Acute PTSD subsides after a duration of three months. If the symptoms persist, the diagnosis is changed to chronic PTSD. The third subform is referred to as delayed onset PTSD which may occur months, years, or even decades after the event.

PTSD first appeared in the Diagnostic and Statistical Manual of Mental Disorders (DSM) in 1980. War veterans are the most publicly-recognized victims of PTSD; long-term psychiatric illness was formally observed in World War I veterans. PTSD has also been recognized as a problem for marginalized groups within societies. One such group is Australian Aboriginal peoples, and other Indigenous peoples around the world. In these cases the repeated history of childhood and adult trauma, removal of children from their families, interpersonal violence and substance abuse, and early death, results in generations of people with high levels of PTSD.

PTSD is normally associated with trauma such as violent crimes, rape, and war experience. However, there have been a growing number of reports of PTSD among cancer survivors and their relatives (Smith 1999, Kangas 2002). Most studies deal with survivors of breast cancer (Green 1998, Cordova 2000, Amir & Ramati, J. Anxiety Disord., 16(2): 195-206, 2002), and cancer in children and their parents (Landolt 1998, Stuber 1998), and show prevalence figures of between five and 20%. Characteristic intrusive and avoidance symptoms have been described in cancer patients with traumatic memories of injury, treatment, and death (Brewin 1998). There is yet disagreement on whether the traumas associated with different stressful events relating to cancer diagnosis and treatment actually qualify as PTSD stressors (Green 1998). Cancer as trauma is multifaceted, includes multiple events that can cause distress, and like combat, is often characterized by extended duration with a potential for recurrence and a varying immediacy of life-threat (Smith 1999).

### Separation Anxiety

Separation Anxiety affects school aged children who struggle to engage socially or participate in the absence of their primary care giver. Separation anxiety can resemble school phobia.

### Exposure Anxiety

Exposure Anxiety was first described in the book, Exposure Anxiety; The Invisible Cage by autistic author Donna Williams and referred to the anxiety associated with feeling one's own existence too extremely to withstand. Exposure Anxiety was described as triggering a pervasive self protective state of involuntary avoidance, diversion and retaliation responses resulting in a struggle to do things ‘as oneself, ‘by oneself or ‘for oneself. By learning to do things as a ‘non-self those with it could sometimes still do things by taking on other characters, roles and voices. Exposure Anxiety was further distinguished from Avoidant Personality Disorder, Oppositional Defiance Disorder and Demand Avoidance Syndrome in the book The Jumbled Jigsaw.

The term “symptom” or “clinical symptom”, as used herein, refers to a subjective evidence of a disease, such as a feeling of nausea, as perceived by the patient. A “sign” refers to objective evidence of a disease as observed by a physician, each elevated blood pressure. Symptoms and signs are not necessarily mutually exclusive.

As used herein, the term “chondroitin sulphate proteoglycans” or “CSPGs” refers to all types of chondroitin sulphate-containing proteoglycans, including but not limited to lecticans and hyalectans, such as neurocan, brevican, aggrecan or versican. “Chondroitin sulfate” is a sulfated glycosaminoglycan (GAG) composed of a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities. Understanding the functions of such diversity in chondroitin sulfate and related glycosaminoglycans is a major goal of glycobiology. Chondroitin sulfate is an important structural component of cartilage and provides much of its resistance to compression.

Chondroitin sulfate was originally isolated well before the structure was characterised, leading to changes in terminology with time. Early researchers identified different fractions of the substance with letters.

Chondroitin sulfate A is sulfated on the carbon 4 of the N-acetylgalactosamine (GalNAc) sugar. Its systematic name is chondroitin-4-sulfate.

Chondroitin sulfate C is sulfated on the carbon 6 of the GalNAc sugar. Its systematic name is chondroitin-4-sulfate chondroitin-6-sulfate.

Chondroitin sulfate D is sulfated on the carbon 2 of the glucuronic acid and 6 of the GalNAc sugar. Its systematic name is chondroitin-4-sulfate chondroitin-2,6-sulfate

Chondroitin sulfate E is sulfated on the carbons 4 and 6 of the GalNAc sugar. Its systematic name is chondroitin-4-sulfate chondroitin-4,6-sulfate

“Chondroitin sulfate B” is an old name for dermatan sulfate, and is no longer classified as a form of chondroitin sulfate.

In chondroitin sulfate, the sulfate is covalently attached to the sugar. Since the molecule has multiple negative charges at physiological pH, a cation is present in salts of chondroitin sulfate.

Chondroitin's functions largely depend on the properties of the overall proteoglycan of which it is a part. These functions can be broadly divided into structural and regulatory roles. However, this division is not absolute and some proteoglycans have both structural and regulatory roles (for example versican).

Chondroitin sulfate is a major component of extracellular matrix, and is important in maintaining the structural integrity of the tissue. This function is typical of the large aggregating proteoglycans: aggrecan, versican, brevican, and neurocan, collectively termed the lecticans. As part of aggrecan, chondroitin sulfate is a major component of cartilage. The tightly packed and highly charged sulfate groups of chondroitin sulfate generate electrostatic repulsion that provides much of the resistance of cartilage to compression. Loss of chondroitin sulfate from the cartilage is a major cause of osteoarthritis.

Chondroitin sulfate readily interacts with proteins in the extracellular matrix due to its negative charges. These interactions are important for regulating a diverse array of cellular activities. The lecticans are a major part of the brain extracellular matrix, where the chondroitin sugar chains function to stabilize normal brain synapses as part of perineuronal nets. The levels of chondroitin sulfate proteoglycans are vastly increased after injury to the central nervous system where they act to prevent regeneration of damaged nerve endings.

The proteoglycan group of C-type lectin-like domains (CTLD)-containing extracellular matrix proteins has four members in both human and mouse, which are also known as lecticans or hyalectans. Neurocan and brevican are expressed in the central nervous system, aggrecan is found principally in cartilage, and versican has a wide tissue distribution. The N-terminal region of a lectican polypeptide consists of an immunoglobulin domain and multiple link modules. The central region, which is divergent and varies significantly in length between different lecticans, serves as an attachment region for glycosaminoglycan chains, primarily chondroitin sulphate. The C-terminal region consists of a CTLD sandwiched between one or two epidermal growth factor (EGF)-like domains and a complement control domain. Brevican is also produced in a truncated, glycosylphosphatidyl inositol-anchored form. High molecular weight aggregates are formed by the binding of multiple lectican polypeptides, through link modules, to a molecule of hyaluronan. Lectican aggregates have structural roles in the extracellular matrix. The resilience of cartilage, which cushions joints, is due to the hydrated glycosaminoglycan chains of aggrecan. Furthermore, the glycosaminoglycan chains and globular domains of lecticans can bind cell surface molecules, matrix components, and extracellular signalling molecules through interactions with protein, lipid or carbohydrate. These interactions are modulated by spatial and temporal variations in lectican expression, splicing, proteolysis, and glycosylation, and by other ligands and signalling molecules. As a consequence, lecticans have complex effects on cell communication, adhesion, migration, proliferation, differentiation and apoptosis. The modulation of these events by lecticans is implicated in development, tissue remodelling, and disease, including cancer and arthritis. The CTLDs in lecticans appear to be of the galactose-binding subtype. The CRD of aggrecan exhibits calcium-dependent binding to galactose and related sugars, but the physiological role of this carbohydrate recognition remains unknown. Lectican CTLDs also participate in protein-protein interactions.

“Proteoglycans” represent a special class of glycoproteins that are heavily glycosylated. They consist of a core protein with one or more covalently attached glycosaminoglycan (GAG) chain(s). These glycosaminoglycan chains are long, linear carbohydrate polymers that are negatively charged under physiological conditions, due to the occurrence of sulfate and uronic acid groups. Proteoglycans can be categorised depending upon the nature of their glycosaminoglycan chains. These chains may be chondroitin sulfate and dermatan sulfate, heparin and heparan sulfate, or keratan sulfate. Proteoglycans can also be categorised by size. Examples of large proteoglycans are aggrecan, the major proteoglycan in cartilage, and versican, present in many adult tissues including blood vessels and skin. The small leucine rich repeat proteoglycans (SLRPs) include decorin, biglycan, fibromodulin and lumican.

As used herein, “chondroitinase” or “CSPGase” refer to any agent able to degrade CSPG in the extracellular matrix. This definition includes, but is not limited to, Chondroitin B lyase, Chondroitin AC lyase, Chondroitin-sulfate-ABC endolyase, N-acetylgalactosamine-6-sulfatase, or Chondro-4-sulfatase. Further examples of chondroitinases suitable for the present invention can also be found in WO-A-2008/045970, WO-A-2005/074655, WO-A-2004/110360, WO-A-2003/074080, WO-A-2003/015612, WO-A-2002/060315, WO-A-1995/029256 or WO-A-1995/029232.

Chondroitin B lyase (EC 4.2.2.19) is an enzyme that catalyzes eliminative cleavage of dermatan sulfate containing 1,4-beta-D-hexosaminyl and 1,3-beta-D-glucurosonyl or 1,3-alpha-L-iduronosyl linkages to disaccharides containing 4-deoxy-beta-D-gluc-4-enuronosyl groups to yield a 4,5-unsaturated dermatan-sulfate disaccharide (deltaUA-GalNAc-4S). This enzyme belongs to the family of lyases, specifically those carbon-oxygen lyases acting on polysaccharides. The systematic name of this enzyme class is chondroitin B lyase. Other names in common use include chondroitinase B, ChonB, and ChnB.

Chondroitin AC lyase (EC 4.2.2.5) is an enzyme that catalyzes the eliminative degradation of polysaccharides containing 1,4-beta-D-hexosaminyl and 1,3-beta-D-glucuronosyl linkages to disaccharides containing 4-deoxy-beta-D-gluc-4-enuronosyl groups. This enzyme belongs to the family of lyases, specifically those carbon-oxygen lyases acting on polysaccharides. The systematic name of this enzyme class is chondroitin AC lyase. Other names in common use include chondroitinase, chondroitin sulfate lyase, chondroitin AC eliminase, chondroitin AC lyase, chondroitinase AC, and ChnAC.

Chondroitin-sulfate-ABC endolyase (EC 4.2.2.20) is an enzyme that catalyzes the endolytic cleavage of beta-1,4-galactosaminic bonds between N-acetylgalactosamine and either D-glucuronic acid or L-iduronic acid to produce a mixture of Delta4-unsaturated oligosaccharides of different sizes that are ultimately degraded to Delta4-unsaturated tetra- and disaccharides. This enzyme belongs to the family of lyases, specifically those carbon-oxygen lyases acting on polysaccharides. The systematic name of this enzyme class is chondroitin-sulfate-ABC endolyase. Other names in common use include chondroitinase, chondroitin ABC eliminase, chondroitinase ABC, chondroitin ABC lyase, chondroitin sulfate ABC lyase, ChS ABC lyase, chondroitin sulfate ABC endoeliminase, chondroitin sulfate ABC endolyase, and ChS ABC lyase I.

N-acetylgalactosamine-6-sulfatase (EC 3.1.6.4) is an enzyme that catalyzes the chemical reaction of cleaving off the 6-sulfate groups of the N-acetyl-D-galactosamine 6-sulfate units of the macromolecule chondroitin sulfate and, similarly, of the D-galactose 6-sulfate units of the macromolecule keratan sulfate. This enzyme belongs to the family of hydrolases, specifically those acting on sulfuric ester bonds. The systematic name of this enzyme class is N-acetyl-D-galactosamine-6-sulfate 6-sulfohydrolase. Other names in common use include chondroitin sulfatase, chondroitinase, galactose-6-sulfate sulfatase, acetylgalactosamine 6-sulfatase, N-acetylgalactosamine-6-sulfate sulfatase, and N-acetylgalactosamine 6-sulfatase. This enzyme participates in glycosaminoglycan degradation and degradation of glycan structures.

Chondro-4-sulfatase (EC 3.1.6.9) is an enzyme that catalyzes the chemical reaction 4-deoxy-beta-D-gluc-4-enuronosyl-(1,3)—N-acetyl-D-galactosamine 4-sulfate+H2O 4-deoxy-beta-D-gluc-4-enuronosyl-(1,3)—N-acetyl-D-galactosamine+sulfate. The 3 substrates of this enzyme are 4-deoxy-beta-D-gluc-4-enuronosyl-(1,3)—N-acetyl-D-galactosamine, 4-sulfate, and H2O, whereas its two products are 4-deoxy-beta-D-gluc-4-enuronosyl-(1,3)—N-acetyl-D-galactosamine and sulfate. This enzyme belongs to the family of hydrolases, specifically those acting on sulfuric ester bonds. The systematic name of this enzyme class is 4-deoxy-beta-D-gluc-4-enuronosyl-(1,3)—N-acetyl-D-galactosamine-4-sulfate 4-sulfohydrolase. This enzyme is also called chondroitin-4-sulfatase.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Examples

### Materials and Methods

### Animals

Adult male C57BL6/J mice (3 months old; Harlan Laboratories, Switzerland) were housed individually for 7 days prior to all experiments. Juvenile C57BL6/J mice were kept in litters together with the dam. All animals were kept under a 12 h light/dark cycle, and provided with food and water ad libitum. All animal procedures were executed in accordance with institutional guidelines and were approved by the Veterinary Department of the Canton of Basel-Stadt.

### Surgery

Mice were anesthetized with isoflurane (induction 5%, maintenance 2.5%) in O₂. Body temperature was maintained with a heating pad (CMA/150, CMA/Microdialysis, Stockholm, Sweden). Mice were secured in a stereotaxic frame and bilaterally implanted in the amygdala with 26 gauge guide cannulae aimed at the following coordinates (1): 1.7 mm posterior to bregma; +/−3.1 mm lateral to midline and 3.0-3.3 mm deep from the cortical surface. The implant was secured using cyanoacrylate adhesive gel. Dummy cannulae were inserted to prevent clogging of the guide cannulae. After surgery mice were allowed to recover for 7 days. Analgesia was applied before, and during 3 days after surgery (Metacam, Boehringer, Basel, Switzerland). At the conclusion of the experiment, injection sites were marked with electrolytic lesions before perfusion and reconstructed with standard histological techniques.

### Drug Infusion

Chondroitinase ABC (chABC) (Sigma) was dissolved in 0.1 M PBS. For infusion of chABC or vehicle (0.1 M PBS), the dummy cannulae were removed and 33-gauge injection cannulae, extending 1 mm below the tip of the guide cannulae, were inserted. The injection cannulae were connected to a microsyringe driven by a microinfusion pump (Stoelting). Animals were given bilateral intra-amygdala injections of 0.2 μl at a constant rate of 0.1 μl/min. The injections cannulae were left in place for 1 min before withdrawal.

### Behavior

Fear conditioning and extinction took place in two different contexts (Context A and B). The conditioning and extinction boxes and the floor were cleaned with 70% ethanol or 1% acetic acid before and after each session, respectively. To score freezing behavior an automatic infrared beam detection system placed on the bottom of the experimental chambers (Coulbourn Instruments, USA) was used. The animals were considered to be freezing if no movement was detected for 2 s. Mice at postnatal day 16 (P16) and 23 (P23) were conditioned using two different protocols in order to match freezing levels. On day 1, P16 and P23 mice were conditioned using 10 (P16) or 5 (P23) pairings of the CS (total CS duration 5 s, white noise, 80 dB) with a US (1 s foot-shock 0.6 mA, inter-trial interval: 20-180 s). The US co-terminated with the CS. On day 2 and day 3, conditioned mice were submitted to extinction training in context B during which they received 12 presentations of the CS on each day. Recall of extinction and context-dependent fear renewal were tested 7 days later in context B and A, respectively, using 4 presentations of the CS.

Twenty four hours after chABC- or vehicle injection, adult mice were submitted to discriminative fear conditioning (day 1) by pairing the CS+ (total CS+duration: 30 s, consisting of 50 ms pips repeated at 0.9 Hz, 2 ms rise and fall, pip frequency: 7.5 kHz, 80 dB) with a US (1 s foot-shock, 0.6 mA, 5 CS+-US pairings; inter-trial interval: 20-180 s). The onset of the US coincided with the offset of the CS+. The CS− was presented after each CS+/US association but was never reinforced (total CS− duration: 30 s, consisting of 50 ms pips repeated at 0.9 Hz, 2 ms rise and fall, pip frequency: white noise, 80 dB, 5 CS− presentations, inter-trial interval: 20-180 s). On days 2 and 3, conditioned mice were submitted to extinction training in context B during which they received 4 and 12 presentations of the CS− and the CS+, respectively. Recall of extinction and context-dependent fear renewal were tested 7 days later in context B and A, respectively, with 4 presentations of the CS− and the CS+.

For evaluation of long-term fear consolidation, chABC- or vehicle-injected mice were conditioned 24 hours after injection by using 5 presentations of the CS (total CS duration: 30 s, consisting of 50 ms pips repeated at 0.9 Hz, 2 ms rise and fall, pip frequency: 7.5 kHz, 80 dB) paired with the delivery of a US (1 s foot-shock 0.6 mA, 5 CS-US pairings, inter-trial interval: 20-180 s). The onset of the US coincided with the offset of the CS. Seven days later, conditioned mice were submitted to a test session in context B during which they received 8 presentations of the CS.

To compare pre-training vs. post-training chABC injections, non injected mice were submitted to a first conditioning session on day 1. Four presentations of the CS1 (total CS duration: 30 s, consisting of 50 ms pips repeated at 0.9 Hz, 2 ms rise and fall, pip frequency: 7.5 kHz, 80 dB) were paired with the delivery of a US (1 s foot-shock 0.6 mA, 4 CS1-US pairings, inter-trial interval: 20-180 s). The onset of the US coincided with the offset of the CS. Conditioned mice were injected with chABC immediately after fear conditioning. Twenty four hours later, they were submitted 24 hours to a test session (day 2) in context B during which they received 8 presentations of the CS1. On day 3 the same mice were submitted to a second conditioning session in which 4 presentations of the CS2 (total CS duration: 30 s, consisting of 50 ms pips repeated at 0.9 Hz, 2 ms rise and fall, pip frequency: white noise, 80 dB) were paired with the delivery of a US (1 s foot-shock 0.6 mA, 4 CS2-US pairings, inter-trial interval: 20-180 s). The onset of the US coincided with the offset of the CS. On day 4, these mice were submitted to two fear test sessions separated by 2 hours in context B during which they received 8 presentations of the CS2 and CS1, respectively.

### Immunohistochemistry

Mice were transcardially perfused with ice-cold 4% paraformaldehyde in 0.1 M phosphate buffered saline and the brains were removed and postfixed in the same solution for 24 hours. Brain sections (80 μm) were cut on a vibratome (VT1000S, Leica Instruments, Switzerland) and incubated for 2 hours in a blocking solution composed of 3% BSA and 0.2% Triton-X-100 in PBS. Sections were incubated overnight at 4° C. in the monoclonal antibody 2b6 (Seikagaku Biobusiness, Japan; 1/200 in PBS) or in a solution containing the biotin-conjugated lectin wisteria floribunda agglutinin (WFA; Vectors Lab, USA 1/200 in PBS). 2b6 staining was detected with a fluorescent dye-coupled goat anti mouse secondary antibody (Alexa Fluor 594, Invitrogen, USA, 1/1000 in PBS). Biotin-conjugated WFA was detected using Cy2-coupled streptavidin (Bio-Concept, Switzerland; 1/1000 in PBS). Stained slices were imaged at 40× using a LSM 510 Meta confocal microscope (Carl Zeiss Inc., Germany). Quantitative analysis of WFA-positive cells was performed using a computerized image analysis system (Imaris 4.2, Bitplane, Switzerland). Structures were defined according to Franklin and Paxinos (1997). Immunoreactive neurons were counted bilaterally using a minimum of 5 sections per hemisphere per animal. Statistical analyses were performed using unpaired Student's t-tests at the P<0.05 level of significance. Results are presented as mean+/−S.E.M.

### Results

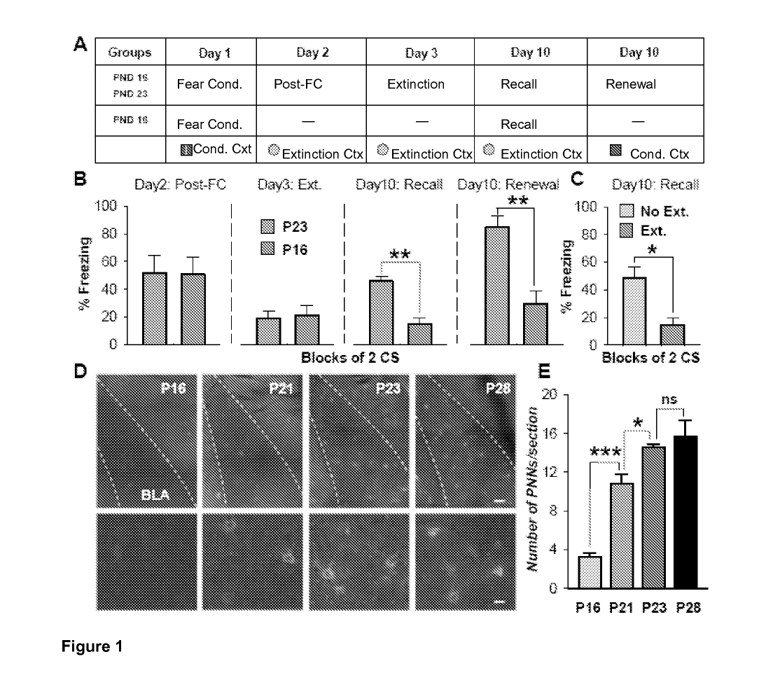

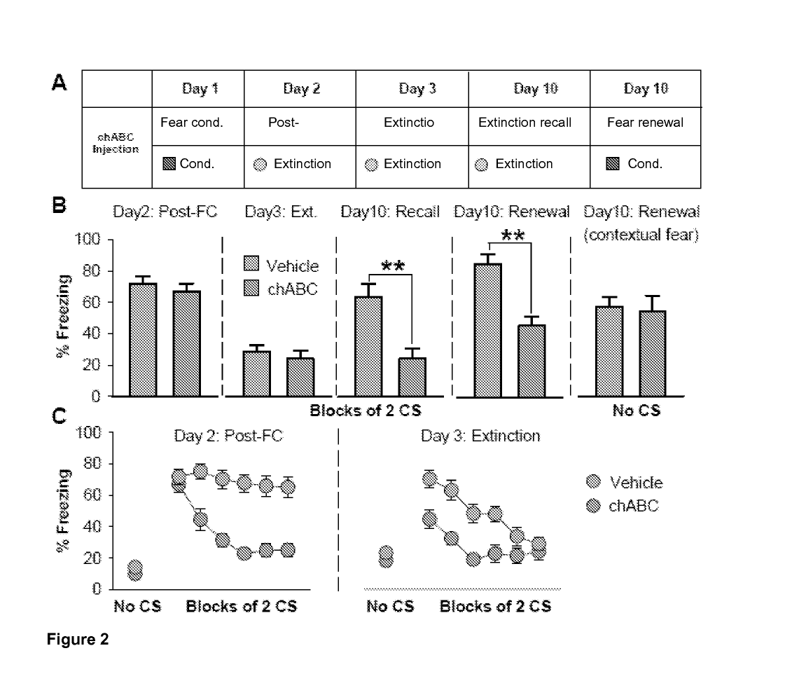

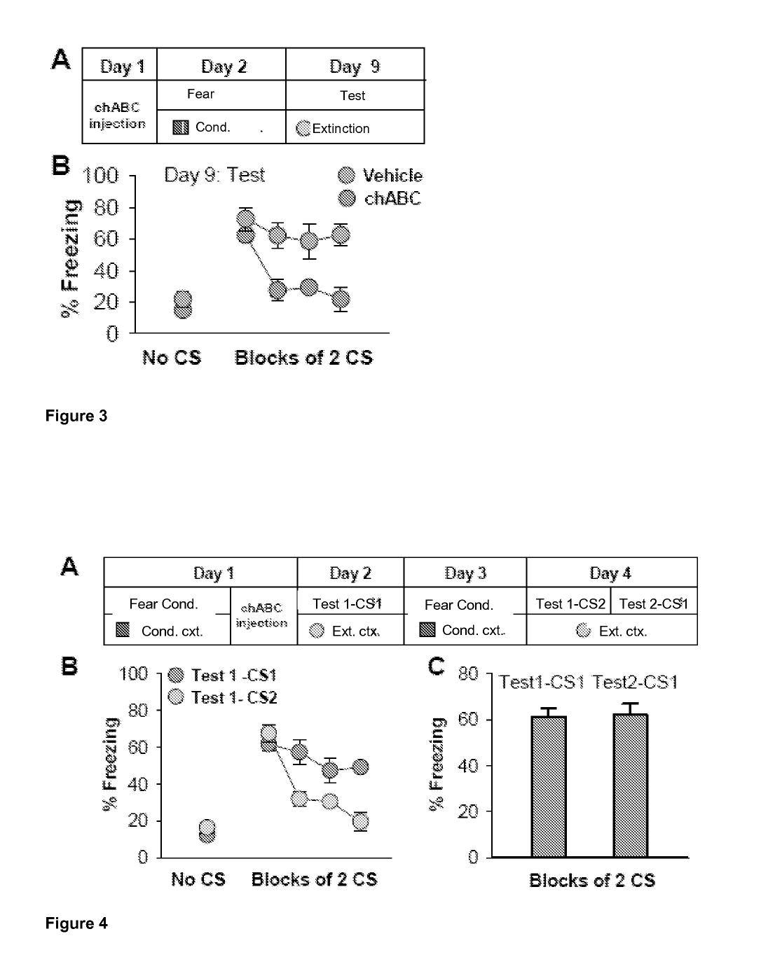

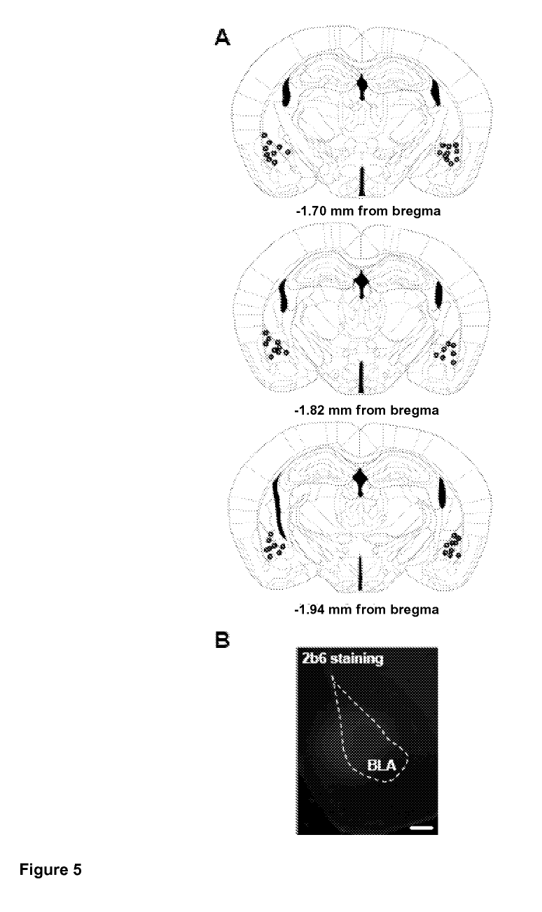

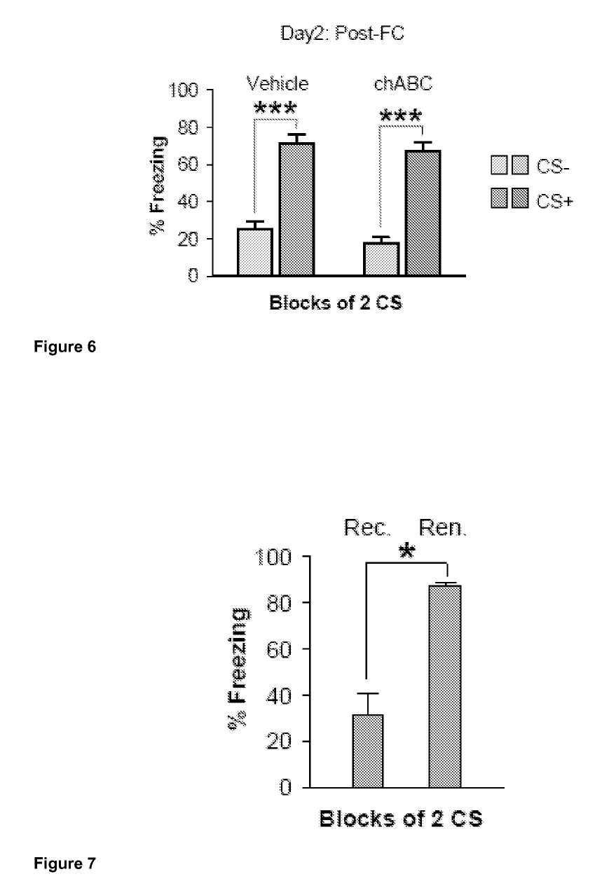

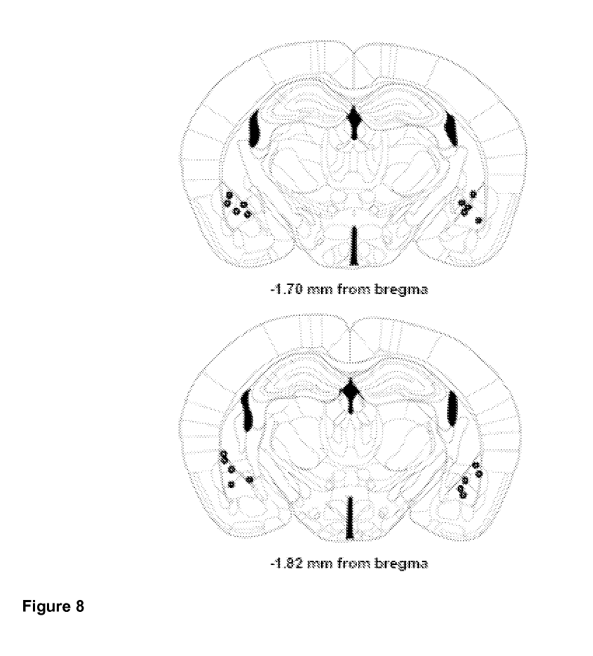

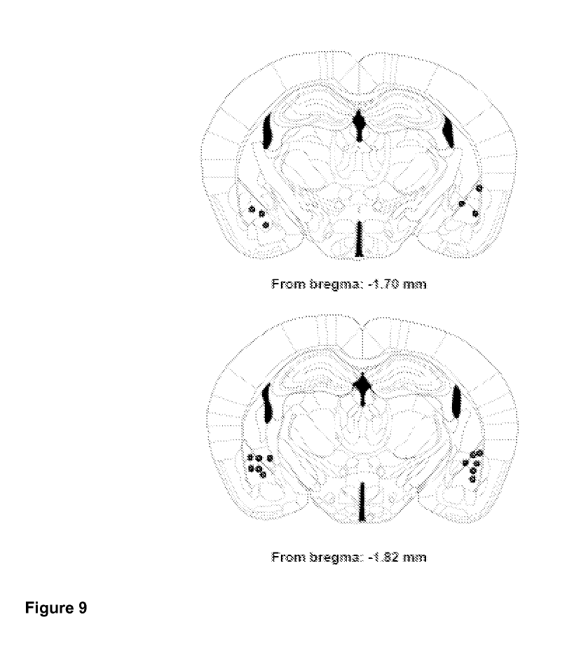

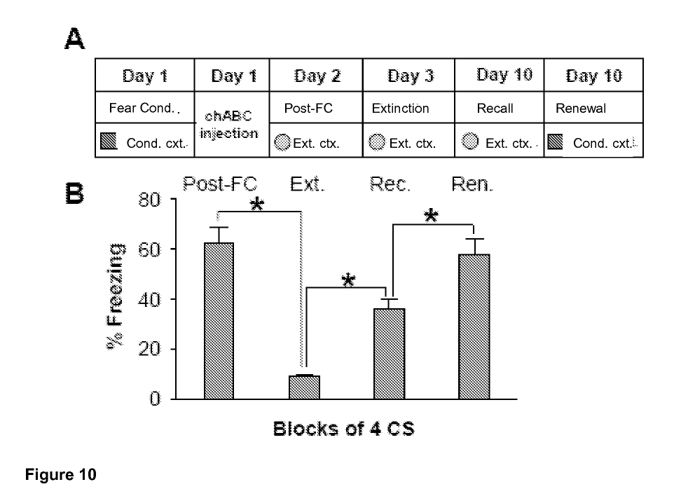

The present inventors first compared spontaneous recovery and context-dependent renewal of extinguished fear responses in young mice conditioned either before (P16) or after (P23) the age of three weeks (FIG. 1A). After fear conditioning, both groups exhibited equal levels of freezing behavior, which was strongly reduced by subsequent extinction training in a different context (FIG. 1B). Seven days after extinction training, mice were re-exposed to the CS in both contexts. Mice conditioned at P23 exhibited significant spontaneous recovery and renewal when tested in the extinction context, or in the fear conditioning context, respectively (FIG. 1B). In contrast, in mice conditioned at P16, freezing levels did not increase compared with those measured at the end of extinction training, independent of the context in which they were exposed to the CS (FIG. 1B). The absence of spontaneous recovery and renewal did not reflect a passive loss of fear memory, as mice conditioned at P16 were able to retain a stable fear memory for 10 days (FIG. 1C). These results extend previous findings from young rats (9, 10) by demonstrating the failure of different manipulations aimed at recovering conditioned fear responses up to one week after extinction learning.

Thus, extinction in mice fear conditioned at about 3 weeks of age appears to reflect new inhibitory learning, whereas extinction in mice fear conditioned just a few days before seems to result in unlearning or erasure of conditioned fear responses.

Next, the present inventors quantified the time course of CSPG expression in the basolateral amygdala (BLA) during the first four postnatal weeks (FIGS. 1D and E). Using the density of wisteria floribunda agglutinin (WFA)-stained PNNs as a read-out for CSPG levels (14), the present inventors found CSPGs to be expressed at low levels and in a diffuse manner at P16 (FIG. 1E). Expression levels and organization into PNNs markedly increased until P28 (FIG. 1E). Notably, the largest increase was observed between P16 and P21 (FIG. 1E), the age around which the switch in the extinction phenotype occurred.

If increased CSPG levels in the BLA were causally related to the development of spontaneous recovery and context-dependent renewal, the present inventors hypothesized that it should be possible to convert the extinction phenotype of adult mice into a juvenile one by acutely degrading CSPGs in adults. To test this, the present inventors locally injected the CSPG-degrading enzyme chondroitinase ABC (chABC) (14, 15) into the BLA of 3 months old mice (FIG. 5). Mice fear conditioned 24 hrs after chABC injection (FIG. 2A) exhibited normal freezing levels compared with vehicle-injected controls when exposed to the CS 24 hrs after conditioning (FIG. 2B). Moreover, both groups exhibited low freezing levels when exposed to an explicitly unpaired CS, indicating that conditioned freezing reflected CS-US associations rather than non-associative sensitization processes (FIG. 6).

This indicates that CSPGs in the BLA are not required for the acquisition, retrieval and expression of conditioned fear memory at this time point.

After extinction training, freezing levels were equally reduced in chABC— and vehicle-injected mice (FIG. 2B). However, when tested 7 days later, a striking difference between the two groups of mice emerged. ChABC-injected mice, like juvenile mice conditioned at P16, exhibited a complete lack of spontaneous recovery and context-dependent renewal (FIGS. 2B and 7). Notably, even though cued (CS-induced) fear behavior was strongly compromised, contextual fear memory appeared to be normal in chABC-injected animals (FIG. 2B). Because contextual fear was not extinguished, extinction training appears to be necessary for the chABC-induced permanent loss of conditioned fear behavior. To examine the time-course of extinction-induced behavioral changes in chABC-injected mice, the present inventors analyzed freezing levels during extinction training. Whereas two extinction training sessions distributed over two days were necessary to achieve full extinction of conditioned fear responses in control animals (FIG. 2C) (16), exposing chABC-injected mice to just 3 CSs was enough to substantially decrease freezing levels, and after 7 CS presentations, freezing behavior was already fully extinguished (FIG. 2C).

Thus, in the absence of CSPGs extinction training triggers a rapid process that results in the acute and permanent loss of conditioned fear behavior. Together, these data demonstrate that degradation of CSPGs in adult mice phenocopies the behavioral consequences of fear extinction in young postnatal animals and suggest that CSPGs prevent unlearning or erasure of fear memories in adults.

Theoretically, there are several alternative mechanisms that may explain the observed lack of conditioned fear responses in chABC-injected mice. Since degraded CSPGs take weeks to turn over after chABC injection (15), one possibility is that degradation of CSPGs could interfere with fear memory consolidation. Although the stability of contextual fear memory in chABC-injected mice suggests otherwise (FIG. 2B), the present inventors specifically tested whether chABC-injected mice were able to form a stable, long-term cued fear memory. Mice were injected with chABC or vehicle (FIG. 8) and fear conditioned 24 hrs later. Long-term fear memory was examined 7 days after conditioning (FIGS. 3A and B). Similar to the 24 hrs time point, chABC— and vehicle-injected mice did not differ in terms of their initial freezing levels even 7 days after fear conditioning (FIG. 3B). Still, repetitive CS exposure induced almost instantaneous extinction in chABC-injected mice, while freezing levels remained constant in control animals (FIG. 3B). This demonstrates that degradation of CSPGs does not affect cued fear memory consolidation, yet allows for rapid extinction of well-consolidated long-term fear memories.

A second possible explanation for the lack of spontaneous recovery and renewal could be that removal of CSPGs enhanced new learning of inhibitory CS-no US associations during extinction training. That is, chABC injections may have strengthened the extinction process per se rather than enabled fear memory erasure. Although difficult to test directly, the present inventors hypothesized that if CSPG degradation acted by strengthening inhibitory learning during extinction training, the effect of chABC injection should be independent of whether animals were injected before or after fear conditioning, as long as CSPGs were degraded before extinction training. They therefore compared extinction of conditioned fear memories acquired before or after chABC injection in the same animals (FIGS. 4A and B, and 9). Degradation of CSPGs after fear conditioning did not accelerate the time course of subsequent extinction learning (FIG. 4B). In the same animals, however, the present inventors observed rapid extinction of a second fear memory acquired in the absence of CSPGs (FIG. 4B). Further, repeated retrieval of fear memories acquired before CSPG degradation revealed stable freezing levels across several days, thus excluding a possible effect on memory reconsolidation, a process that occurs in parallel and competes with extinction when memories are repeatedly retrieved during extinction training (17) (FIG. 4C). Finally, the present inventors examined whether fear memories acquired before chABC injection could be extinguished normally. Mice fear conditioned 24 hrs before chABC injection exhibited normal extinction, and, when tested one week later, showed normal levels of spontaneous recovery and context-dependent renewal (FIG. 10).

Together, these findings demonstrate that CSPG degradation does not strengthen new inhibitory learning during extinction or impair memory reconsolidation, but renders fear memory traces susceptible to unlearning or erasure. Because chABC injection had only anterograde, but no retrograde effects, the present inventors conclude that the state of fear memories acquired in the absence of CSPGs fundamentally differs compared with the state of memories acquired in the presence of CSPGs. Whereas in the former state repeated non-reinforced CS presentations lead to the active inhibition of conditioned fear responses, in the latter state the memory trace is erased.

### Discussion

What might be the mechanism by which degradation of CSPGs enables fear memory erasure? Fear memories are acquired and stored, at least in part, by learning-induced strengthening of synaptic inputs to the lateral (LA) or basal (BA) nucleus of the amygdala through N-methyl-D-aspartate (NMDA) receptor-dependent long-term potentiation (LTP) (1, 18, 19). Extinction training, in turn, is thought to leave this memory trace largely intact, but to suppress amygdala output by recruiting inhibitory networks (3-5, 20-22). However, it has been suggested that fear memories may be unlearned, rather than suppressed, if extinction occurs immediately after fear conditioning, possibly involving LTP reversal, so-called depotentiation (23, 24). Thus, without wishing to be bound by theory, one possible mechanism could be that CSPGs prevent fear memory erasure by rendering potentiated synapses resistant to depotentiation.

An alternative possibility relates to the finding that CSPG-containing PNNs are primarily colocalized with parvalbumin (PV)-positive GABAergic interneurons (13), a major subpopulation of inhibitory interneurons in the BLA (25). Functional maturation of inhibitory networks containing PV-positive interneurons underlies critical period opening during visual cortex development (12). Moreover, treatment of cultured hippocampal PV-positive interneurons with chABC increased interneuron excitability (26). Thus, without wishing to be bound by theory, degradation of CSPGs may alter the function of local inhibitory circuits in such a way that fear conditioning leads to the formation of an erasure-prone memory trace.

Because context-dependent renewal of conditioned fear responses is believed to be an important factor contributing to the relapse of pathological fear in patients undergoing therapy for anxiety disorders (27), the present findings provide for novel strategies in preventing the development of extinction-resistant pathological fear and anxiety.

In keeping with studies on young postnatal rats (9, 10), the present study indicates that qualitatively distinct neuronal mechanisms mediate extinction learning during early postnatal development and in adults. However, since resistance to extinction-induced memory erasure critically depends on the presence of CSPGs at the time point of fear conditioning, it is likely that not only fear extinction, but the neuronal substrates of fear conditioning and, as a consequence, the representation of fear memories are also developmentally regulated. Indeed, a first developmental switch in the neuronal circuitry mediating aversive conditioning occurs around P10, when young rodents start to exhibit exploratory behavior. Whereas before P10, aversive conditioning results in the paradoxical acquisition of an amygdala-independent approach behavior, amygdala-dependent conditioned fear behavior can only be acquired by animals older than 9 days (8). Thus, once young rodents start to explore their environment, the need for predicting aversive and potentially life-threatening events is met by the functional maturation of the neuronal circuitry of fear conditioning. However, because several components of the neuronal circuitry mediating fear extinction in adults, including the connections between the amygdala and the mPFC (6, 7) as well as local inhibitory circuits (28), are not yet functionally mature at this age, this creates a critical period during which conditioned fear behavior cannot be extinguished. This may have detrimental consequences on adult emotional behavior and learning (29). The present findings indicate that the delayed maturation of extracellular matrix CSPGs represents a protective mechanism enabling the erasure of aversive memories during this early postnatal critical period.

### REFERENCES

1. J. E. LeDoux, Annu. Rev. Neurosci. 23, 155 (2000).
2. G. D. Gale et al., J. Neurosci. 24, 3810 (2004).
3. K. M. Myers, M. Davis, Mol. Psychiatry 12, 120 (2007).
4. M. E. Bouton, R. F. Westbrook, K. A. Corcoran, S. Maren, Biol. Psychiatry 60, 352 (2006).
5. G. J. Quirk, R. Garcia, F. Gonzalez-Lima, Biol. Psychiatry 60, 337 (2006).
6. H. Bouwmeester, K. Smits, J. M. van Ree, J. Comp. Neurol. 450, 241 (2002).
7. M. G. Cunningham, S. Bhattacharyya, F. M. Benes, J. Comp. Neurol. 453, 116 (2002).
8. R. M. Sullivan, D. A. Wilson, C. Lemon, G. A. Gerhardt, Nature 407, 38 (2000).
9. J. H. Kim, R. Richardson, Behav. Neurosci. 121, 131 (2007).
10. J. H. Kim, R. Richardson, Neurobiol. Learn. Mem. 88, 48 (2007).
11. J. H. Kim, R. Richardson, J. Neurosci. 28, 1282 (2008).
12. T. K. Hensch, Nat. Rev. Neurosci. 6, 877 (2005).
13. C. M. Galtrey, J. W. Fawcett, Brain Res. Rev. 54, 1 (2007).
14. T. Pizzorusso et al., Science 298, 1248 (2002).
15. G. Brückner et al., Exp. Brain Res. 121, 300 (1998).
16. C. Herry et al., Nature 454, 600 (2008).
17. M. Eisenberg, T. Kobilo, D. E. Berman, Y. Dudai, Science 301, 1102 (2003).
18. S. Maren, G. J. Quirk, Nat. Rev. Neurosci. 5, 844 (2004).
19. D. Anglada-Figueroa, G. J. Quirk, J. Neurosci. 25, 9680 (2005).
20. J. A. Rosenkranz, A. A. Grace, J. Neurosci. 77, 489 (2003).
21. D. Paré, G. J. Quirk, J. E. LeDoux, J. Neurophysiol. 92, 1 (2004).
22. E. Likhtik et al., Nature 454, 642 (2008).
23. K. M. Myers, K. J. Ressler, M. Davis, Learn. Mem. 13, 216 (2006).
24. C. H. Lin, P. W. Gean, Mol. Pharmacol. 63, 44 (2003).
25. A. J. McDonald, F. Mascagni, Neuroscience 105, 681 (2001).
26. A. Dityatev et al., Develop. Neurobiol. 67, 570 (2007).
27. B. I. Rodriguez, M. G. Craske, S. Mineka, D. Hladek, Behav. Res. Ther. 37, 845 (1999).
28. B. Berdel, J. Morys, Int. J. Devl. Neuroscience 18, 501 (2000).
29. C. R. Pryce, J. Feldon, Neurosci. Biobehay. Rev. 27, 57 (2003).

1. A method of modulating, treating or effecting prophylaxis of a subject having anxiety or at risk of developing symptoms of anxiety, said method being characterized in that a therapeutically effective amount of a modulator of chondroitin sulphate proteoglycans is administered to said subject.

2. The method of claim 1 wherein the modulator degrades the chondroitin sulphate proteoglycans of the extracellular matrix.

3. The method of claim 1 wherein the modulator modulates the expression, translation and/or secretion of the chondroitin sulphate proteoglycans to the extracellular matrix.

4. The method of claim 1, wherein the modulator decreases or increases the interaction of the chondroitin sulphate proteoglycans of the extracellular matrix with their counterparts, and/or modifies the molecular organization of the chondroitin sulphate proteoglycans of the extracellular matrix.

5. The method of claim 1, wherein the modulator of is a small molecule.

6. The method of claim 1, wherein the modulator is an antibody.

7. The method of claim 1, wherein the modulator is a peptide.

8. The method of claim 1, wherein the modulator decreases or silences the expression of chondroitin sulphate proteoglycans or of a negative regulator thereof.

9. The method of claim 1, wherein the modulator is a siRNA.

10. The method of claim 1, wherein the modulator is an enzyme.

11. The method of claim 1, wherein the subject is a mammal.

12. The method of claim 1, wherein the modulator is delivered or applied to the amygdala of the subject.

13. A siRNA modulating the expression, translation and/or secretion of the chondroitin sulphate proteoglycans to the extracellular matrix.

14. An antibody specifically binding to chondroitin sulphate proteoglycans.

15. An enzyme degrading chondroitin sulphate proteoglycans.

16. The method of claim 10, wherein the enzyme is chondroitinase.

17. The method of claim 11, wherein the mammal is a human.

18. The enzyme of claim 15, wherein the enzyme is a chondroitinase.
